(19) United States
(12) Patent Application Publication
(10) Pub. No.: US 2025/0375094 A1
(43) Pub. Date: Dec. 11, 2025

(54) ENDOSCOPE SYSTEM AND INTERFACE ADAPTER

(71) Applicant: FUJIFILM Corporation, Tokyo (JP)

(72) Inventors: Teppei YAMAKI, Ashigarakami-gun (JP); Takahiro MISHIMA, Ashigarakami-gun (JP)

(73) Assignee: FUJIFILM Corporation, Tokyo (JP)

(21) Appl. No.: 19/300,687

(22) Filed: Aug. 15, 2025

Related U.S. Application Data
(63) Continuation of application No. PCT/JP2023/045789, filed on Dec. 20, 2023.

(30) Foreign Application Priority Data
Feb. 22, 2023 (JP) 2023-025924

(51) Int. Cl. A61B 1/00; A61B 1/045; A61B 1/06; G06T 5/90

(52) U.S. Cl. CPC A61B 1/000095; A61B 1/00048; A61B 1/00124; A61B 1/045; A61B 1/0655; G06T 5/90; G06T 2207/10068; G06T 2207/20172

(57) ABSTRACT

An interface adapter includes: a first communication interface for communicating with a scope including an imaging sensor and a light source device that generates illumination light for imaging by the imaging sensor; a second communication interface for communicating with a portable information terminal; and a processor, and the processor causes the imaging sensor to perform imaging in a state in which a light amount of the illumination light is controlled to be constant, converts a captured image signal obtained by the imaging sensor into image data that is displayable by the portable information terminal, and transmits the image data to the portable information terminal, derives a brightness of the image data based on the captured image signal, and controls an imaging sensitivity of the imaging sensor and an exposure time of the imaging sensor based on the brightness.

### CROSS REFERENCE TO RELATED APPLICATION

This is a continuation of International Application No. PCT/JP2023/045789 filed on Dec. 20, 2023, and claims priority from Japanese Patent Application No. 2023-025924 filed on Feb. 22, 2023, the entire disclosure of which is incorporated herein by reference.

### BACKGROUND OF THE INVENTION

### 1. Field of the Invention

The present invention relates to an endoscope system and an interface adapter.

### 2. Description of the Related Art

In recent years, disposable endoscopes that can be used safely from the viewpoint of hygiene for a single use have been receiving attention. JP2012-511357A describes an endoscope in which an image signal is transmitted from a camera of the endoscope to a control operation unit, and the image signal is sent from the control operation unit to a video display via an electric wire. JP2020-18876A describes a laparoscope system in which a laparoscope communicates with a dongle and the dongle transmits image data to a television display.

### SUMMARY OF THE INVENTION

An object of the present disclosure is to construct a system capable of displaying an image based on an imaging signal obtained by a scope on a general-purpose portable information terminal at a low cost.

An interface adapter according to an aspect of the present disclosure comprises: a first communication interface for communicating with a scope including an imaging sensor and a light source device that generates illumination light for imaging by the imaging sensor; a second communication interface for communicating with a portable information terminal; and a processor, in which the processor causes the imaging sensor to perform imaging in a state in which a light amount of the illumination light is controlled to be constant, converts a captured image signal obtained by the imaging sensor into image data that is displayable by the portable information terminal, and transmits the image data to the portable information terminal, derives a brightness of the image data based on the captured image signal, and controls an imaging sensitivity of the imaging sensor and an exposure time of the imaging sensor based on the brightness.

According to the present disclosure, it is possible to construct a system capable of displaying an image based on an imaging signal obtained by a scope on a general-purpose portable information terminal at a low cost.

### BRIEF DESCRIPTION OF THE DRAWINGS

FIG. 1 is a diagram showing an example of an endoscope system 100 according to an embodiment of the present invention.

FIG. 2 is a diagram (Part 1) showing an example of an interface adapter 4.

FIG. 3 is a diagram (Part 2) showing an example of the interface adapter 4.

FIG. 4 is a diagram (Part 1) showing an example of a state before the interface adapter 4 and a portable information terminal 7 are accommodated in a frame 8.

FIG. 5 is a diagram (Part 2) showing an example of the state before the interface adapter 4 and the portable information terminal 7 are accommodated in the frame 8.

FIG. 6 is a diagram (Part 1) showing an example of a state in which the interface adapter 4 and the portable information terminal 7 are attached to a rear case 82.

FIG. 7 is a diagram (Part 2) showing an example of the state in which the interface adapter 4 and the portable information terminal 7 are attached to the rear case 82.

FIG. 8 is a diagram (Part 1) showing an example of a state in which a front cover 81 is attached to the rear case 82.

FIG. 9 is a diagram (Part 2) showing an example of a state in which the front cover 81 is attached to the rear case 82.

FIG. 10 is a diagram showing an example of an internal configuration of a scope 1 and the interface adapter 4.

FIG. 11 is a diagram showing an example of a hardware configuration of the portable information terminal 7.

FIG. 12 is a flowchart for describing a detailed example of metering processing and exposure control performed by a system control unit 44.

FIG. 13 is a schematic diagram for describing a relationship between a magnitude of an exposure change amount ΔEV[Log], an exposure time SS, and an amplification factor DG.

FIG. 14 is a schematic diagram for describing gamma correction processing.

FIG. 15 is a schematic diagram showing an example of an image displayed on a display unit 7a in a case where the scope 1 is a bronchoscope.

FIG. 16 is a diagram showing a display example of image data subjected to the gamma correction to have output gradation characteristics of a solid line shown in a graph 53.

FIG. 17 is a diagram showing an example of a gain correction table.

### DESCRIPTION OF THE PREFERRED EMBODIMENTS

Hereinafter, an embodiment of the present invention will be described with reference to the drawings.

### Endoscope System 100 According to Embodiment of Present Invention

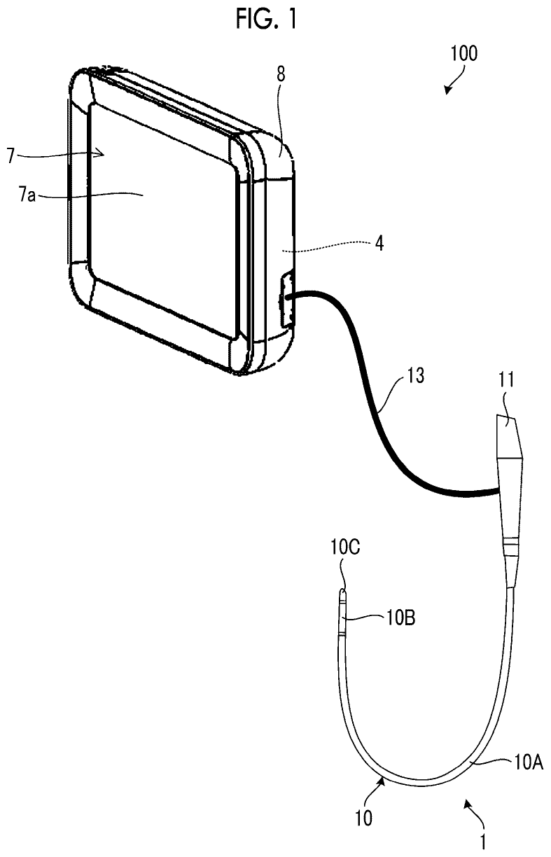

FIG. 1 is a diagram showing an example of an endoscope system 100 according to an embodiment of the present invention. The endoscope system 100 includes a scope 1, an interface adapter 4 connected to the scope 1, and a frame 8 that accommodates a portable information terminal 7 connected to the interface adapter 4. In FIG. 1, the interface adapter 4 is located behind the portable information terminal 7 and is covered by the frame 8, and is therefore not visible. The endoscope system 100 may further include the portable information terminal 7.

The scope 1 is an endoscope including an insertion part 10 that is a tubular member extending in one direction and that is inserted into a subject, and an operation part 11 that is provided at a proximal end portion of the insertion part 10 and that is used to perform various operations of the scope 1. The operation part 11 includes, for example, an angle knob that bends the insertion part 10 by a rotational movement operation. In addition, the operation unit 11 may include an operation member for performing an observation mode switching operation, an imaging and recording operation, a forceps operation, an air/water supply operation, a suction operation, and the like of the scope 1.

The scope 1 is connected to the interface adapter 4 via a communication cable 13. In addition, the scope 1 is attachable to and detachable from the interface adapter 4 via the communication cable 13, and can be used once (that is, disposable). For example, the communication cable 13 may be attachable to and detachable from the interface adapter 4, or the scope 1 may be attachable to and detachable from the communication cable 13.

In addition, although omitted in FIG. 1, various channels, such as a forceps hole for inserting forceps for sampling a biological tissue, such as cells or polyps, an air and water supply channel, and a suction channel, are provided inside the operating part 11 and the insertion part 10.

The insertion part 10 is composed of a flexible part 10A that has flexibility, a bending part 10B provided at a distal end of the flexible part 10A, and a hard distal end part 10C provided at a distal end of the bending part 10B. The bending part 10B is configured to be bendable via an operation of the operating part 11 (for example, the angle knob). The bending part 10B can bend in any direction and at any angle depending on a site of the subject for which the scope 1 is used, allowing the distal end part 10C to be directed in a desired direction.

The interface adapter 4 connects the scope 1 and the portable information terminal 7. Specifically, the interface adapter 4 is connected to the scope 1 via, for example, a communication cable 13 in a communicable manner. In addition, the interface adapter 4 is communicatively connected to the portable information terminal 7 in a wired or wireless manner.

In addition, the interface adapter 4 receives an imaging signal obtained by imaging an inside of the subject with an imaging sensor of the scope 1 from the scope 1, and converts the received imaging signal into image data that can be displayed by the portable information terminal 7. Then, the interface adapter 4 transmits the converted image data to the portable information terminal 7.

The portable information terminal 7 is a general-purpose portable information terminal such as a tablet terminal or a smartphone. The portable information terminal 7 has a display unit 7a that can display an image based on the image data. The portable information terminal 7 receives a captured image or the like obtained by imaging the inside of the subject with the scope 1 from the interface adapter 4 and displays the received captured image on the display unit 7a. The display unit 7a has a display surface on which display pixels are two-dimensionally arranged, and pixel data constituting image data is drawn on each display pixel on the display surface, thereby displaying the image based on the image data. In addition, the portable information terminal 7 also serves as a user interface for controlling the interface adapter 4.

### Interface Adapter 4

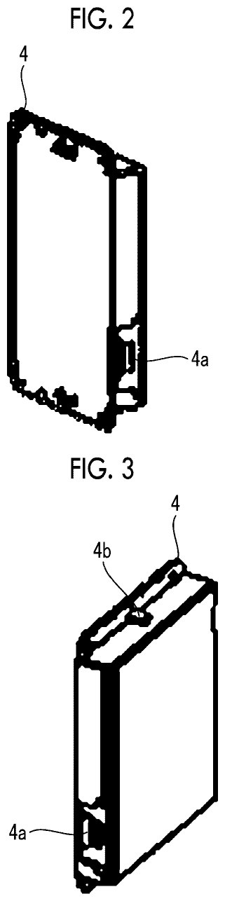

FIGS. 2 and 3 are diagrams showing an example of the interface adapter 4. FIGS. 2 and 3 are diagrams of the interface adapter 4 as viewed in different directions. As shown in FIGS. 2 and 3, the interface adapter 4 has a substantially rectangular parallelepiped shape and has a video input terminal 4a and a video output terminal 4b.

Each of the video input terminal 4a and the video output terminal 4b can be a terminal of various communication standards capable of transmitting a video signal, such as a Universal Serial Bus (USB), a High-Definition Multimedia Interface (HDMI), and a Digital Visual Interface (DVI). HDMI is a registered trademark.

The video input terminal 4a is a terminal for communicative connection to the scope 1. For example, the video input terminal 4a is an HDMI terminal, and the scope 1 is also provided with an HDMI terminal. The communication cable 13 is an HDMI cable, and the HDMI terminals of the video input terminal 4a and the scope 1 are connected to each other by the communication cable 13.

However, the connection between the interface adapter 4 and the scope 1 is not limited to an HDMI connection, and may be a wired connection other than HDMI, such as USB or DVI, or a wireless connection via Bluetooth, short-range wireless communication, or the like. Bluetooth is a registered trademark. In addition, the interface adapter 4 and the scope 1 may be connected to each other via a conversion adapter that converts communication standards.

The video output terminal 4b is a terminal for communicative connection to the portable information terminal 7. For example, the video output terminal 4b is a USB terminal, and the portable information terminal 7 is also provided with a USB terminal. The USB terminals of the video output terminal 4b and the portable information terminal 7 are connected to each other by a USB cable.

However, the connection between the interface adapter 4 and the portable information terminal 7 is not limited to a USB connection, and may be a wired connection other than USB, such as HDMI or DVI, or a wireless connection via Bluetooth, short-range wireless communication, or the like. In addition, the interface adapter 4 and the portable information terminal 7 may be connected to each other via a conversion adapter that converts communication standards.

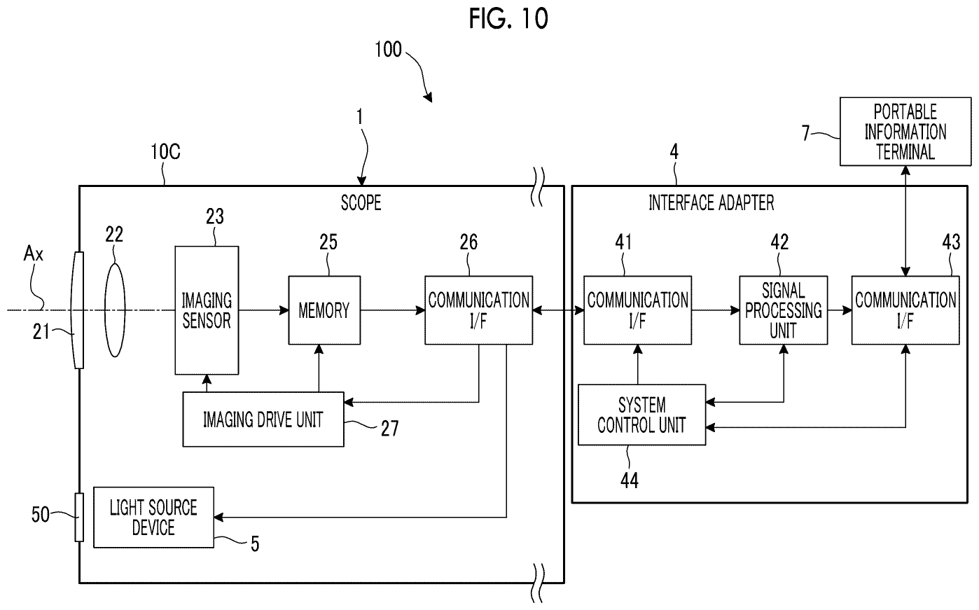

In addition, a circuit that controls the scope 1 or converts the imaging signal from the scope 1 into image data that can be displayed by the portable information terminal 7 is provided inside the interface adapter 4. An internal configuration of the interface adapter 4 will be described later (for example, see FIG. 10).

### State Before Accommodation of Interface Adapter 4 and Portable Information Terminal 7 in Frame 8

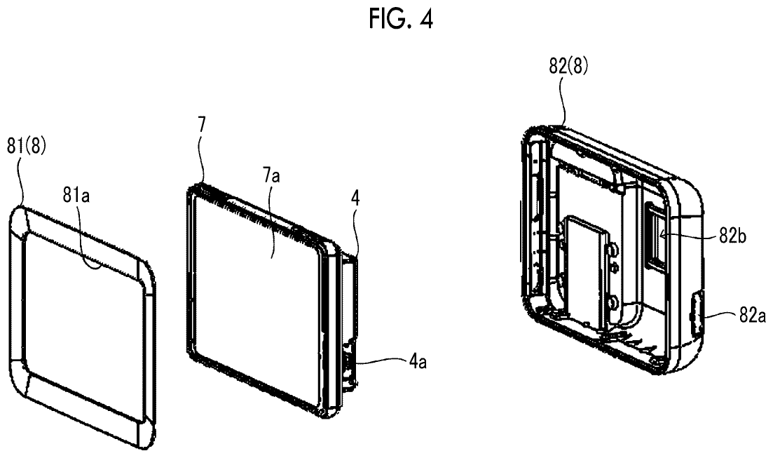

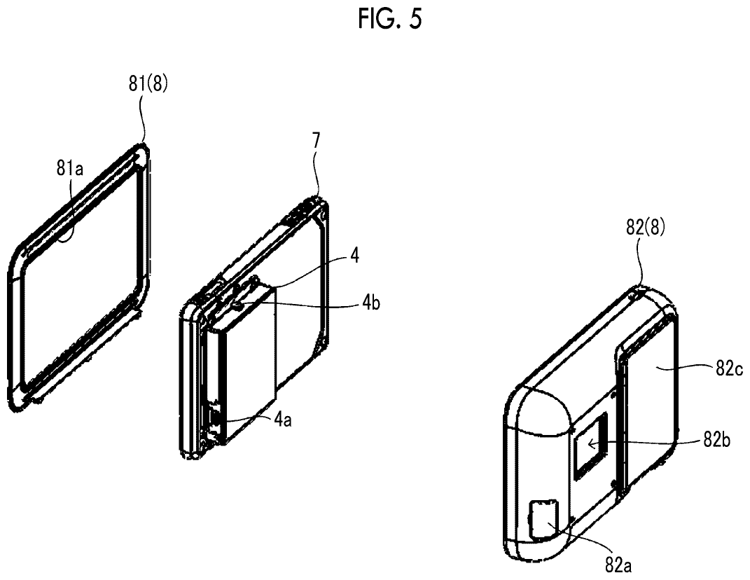

FIGS. 4 and 5 are diagrams showing an example of a state before the interface adapter 4 and the portable information terminal 7 are accommodated in the frame 8. FIGS. 4 and 5 are diagrams showing the interface adapter 4, the portable information terminal 7, and the frame 8 as viewed in different directions. As shown in FIGS. 4 and 5, the frame 8 includes a front cover 81 and a rear case 82.

The front cover 81 is a member that protects a front surface of the portable information terminal 7. The front cover 81 has an opening portion 81a for exposing the display unit 7a through the front cover 81 such that the display unit 7a (touch panel) of the portable information terminal 7 can be displayed and a touch operation can be performed on the display unit 7a.

The rear case 82 is a member that protects a rear surface of the portable information terminal 7. In addition, the rear case 82 is provided with a cable insertion hole (not shown) that allows the communication cable 13 to be connectable to the video input terminal 4a of the interface adapter 4 accommodated in the frame 8 from an outside of the frame 8, and the rear case 82 is provided with a cable insertion hole cover 82a that closes the cable insertion hole. In addition, the rear case 82 is provided with a notation viewing hole 82b so that a notation such as standard compliance on a housing of the interface adapter 4 can be visually recognized from the outside of the frame 8.

The interface adapter 4 is accommodated in the frame 8 together with the portable information terminal 7, whereby the interface adapter 4 is fixed to the frame 8. In the examples of FIGS. 4 and 5, the interface adapter 4 is attached to a back surface of the portable information terminal 7 fixed by the frame 8, and is fixed to the frame 8 via the portable information terminal 7.

The interface adapter 4 is attached to the portable information terminal 7 by, for example, screwing using a screw hole provided on the back surface of the portable information terminal 7. In addition, the interface adapter 4 may be attached to the portable information terminal 7 via a sheet metal or the like formed in accordance with a shape of a back surface of the interface adapter 4.

In addition, the interface adapter 4 is not limited to the configuration in which the interface adapter 4 is fixed to the frame 8 via the portable information terminal 7, and the interface adapter 4 may be directly attached to the frame 8. For example, the interface adapter 4 may be attached to the rear case 82 of the frame 8.

In addition, a stand 82c may be provided on an outside of the rear case 82. The stand 82c is provided on the rear case 82 via, for example, a hinge, and by pulling out a part of the stand 82c from the rear case 82, the frame 8 can stand on a horizontal surface such as a tabletop.

In addition, the rear case 82 may be provided with a screw hole that can be used in a case of attaching the frame 8 to a wall-mount bracket, an arm, a stand, or the like, in addition to the stand 82c or instead of the stand 82c. The screw hole can be configured to comply with, for example, a Video Electronics Standards Association (VESA) standard.

In the state of FIGS. 4 and 5, the video output terminal 4b of the interface adapter 4 and the portable information terminal 7 are connected to each other via a communication cable (for example, a USB cable).

### State in Which Interface Adapter 4 and Portable Information Terminal 7 Are Attached to Rear Case 82

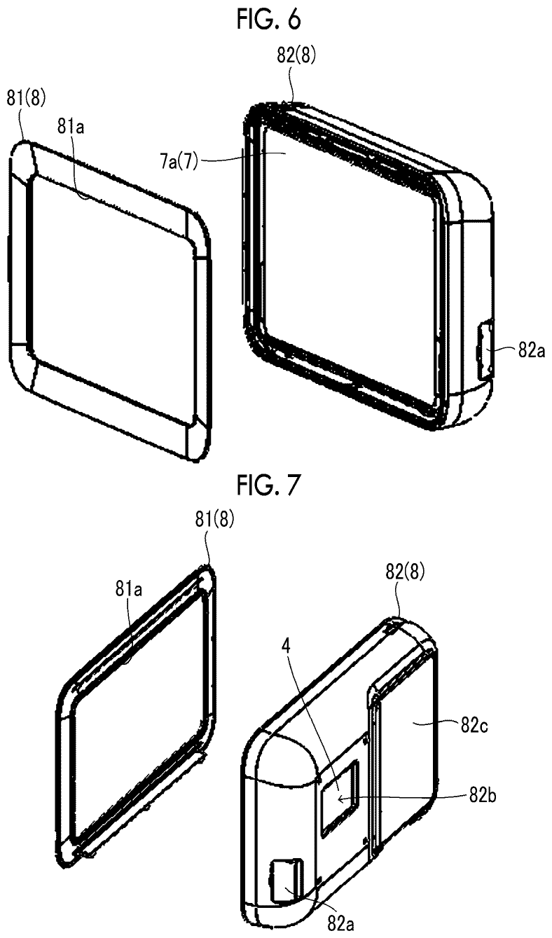

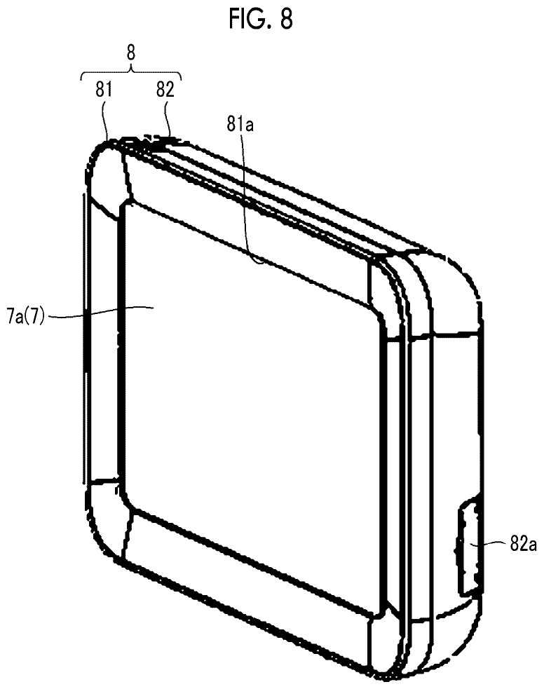

FIGS. 6 and 7 are diagrams showing an example of a state in which the interface adapter 4 and the portable information terminal 7 are attached to the rear case 82. FIGS. 6 and 7 are diagrams showing the interface adapter 4, the portable information terminal 7, and the frame 8 as viewed in different directions.

In the state in which the interface adapter 4 is attached to the back surface of the portable information terminal 7 as shown in FIGS. 4 and 5, the portable information terminal 7 and the interface adapter 4 are attached to an inside of the rear case 82 as shown in FIGS. 6 and 7. Accordingly, the notation such as standard compliance on the housing of the interface adapter 4 is exposed through the notation viewing hole 82b. In addition, by removing the cable insertion hole cover 82a, the video input terminal 4a of the interface adapter 4 enters a state of being exposed through the cable insertion hole of the rear case 82.

### State in Which Front Cover 81 is Attached to Rear Case 82

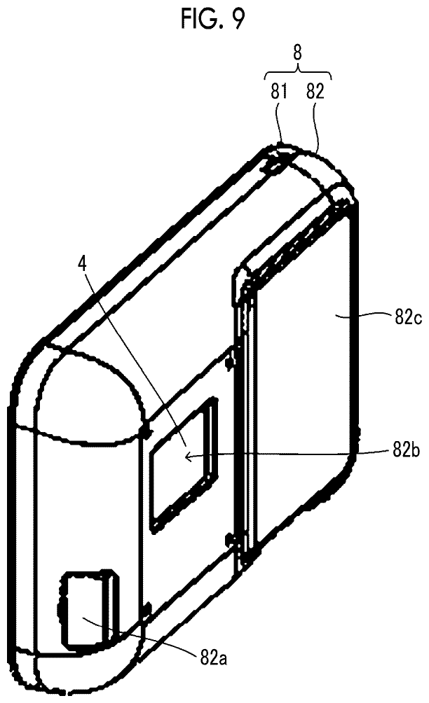

FIGS. 8 and 9 are diagrams showing an example of a state in which the front cover 81 is attached to the rear case 82. FIGS. 8 and 9 are diagrams of the frame 8 and the like as viewed in different directions.

In the state in which the portable information terminal 7 and the interface adapter 4 are attached to the rear case 82 as shown in FIGS. 6 and 7, the front cover 81 is attached to the rear case 82 as shown in FIGS. 8 and 9. Accordingly, the portable information terminal 7 and the interface adapter 4 are accommodated in the frame 8.

The frame 8 has a waterproof structure to prevent water, dust, and the like from infiltrating from the outside in the state shown in FIGS. 8 and 9. For example, a packing for filling a joint portion between the front cover 81 and the rear case 82 is provided between the front cover 81 and the rear case 82. In addition, a frame portion of the opening portion 81a of the front cover 81 is sealed to be in close contact with the display unit 7a of the portable information terminal 7, and the opening portion 81a is closed by the display unit 7a.

In addition, a frame portion of the notation viewing hole 82b of the rear case 82 is sealed to be in close contact with the housing of the interface adapter 4, and the notation viewing hole 82b is closed by the housing of the interface adapter 4.

In addition, a frame portion of the cable insertion hole of the rear case 82 is sealed to be in close contact with the housing of the interface adapter 4, and in a case where the cable insertion hole cover 82a is removed from the rear case 82, the cable insertion hole of the rear case 82 is closed by the housing of the interface adapter 4. Further, in a case where the cable insertion hole cover 82a is removed from the rear case 82, a periphery of the cable insertion hole of the rear case 82 is sealed such that the cable insertion hole of the rear case 82 is closed by the communication cable 13 inserted through the cable insertion hole of the rear case 82.

That is, the frame 8 has a waterproof structure to prevent water, dust, and the like from entering an inside of the frame 8 by using the interface adapter 4 and the portable information terminal 7 accommodated therein. Accordingly, even in a case where the portable information terminal 7, the interface adapter 4, and the frame 8 are used in an environment such as outdoors, it is possible to prevent water, dust, and the like from entering the inside of the frame 8 and to protect the interface adapter 4 and the portable information terminal 7.

As described above, the endoscope system 100 has a configuration in which the interface adapter 4 that converts an imaging signal obtained by an imaging sensor 23 of the scope 1 into image data that can be displayed on the portable information terminal 7 is fixed to the frame 8 that accommodates the portable information terminal 7.

Accordingly, since the interface adapter 4 performs processing of converting the imaging signal obtained by the imaging sensor 23 of the scope 1 into image data, the portable information terminal 7 can be a general-purpose portable information terminal such as a tablet terminal. Therefore, it is easy to procure, replace, and update the portable information terminal 7.

In addition, since the interface adapter 4 is fixed to the frame 8 that accommodates the portable information terminal 7, the portable information terminal 7 and the interface adapter 4 are integrated by the frame 8. Therefore, it is possible to facilitate handling in a case where the scope 1 is operated while observing the image based on the imaging signal obtained by the imaging sensor 23 of the scope 1 using the portable information terminal 7.

For example, a user (for example, a doctor) of the endoscope system 100 can operate the scope 1 while observing the image without concern for a position of the interface adapter 4 between the scope 1 and the portable information terminal 7. In addition, it is possible to prevent an accident such as the interface adapter 4 between the scope 1 and the portable information terminal 7 falling and pulling the scope 1 in a case where the user operates the scope 1 while observing the image.

In addition, by providing the interface adapter 4 between the scope 1 and the portable information terminal 7, an image processing circuit (for example, a signal processing unit 42) that converts the imaging signal obtained by the imaging sensor 23 of the scope 1 into image data need not be provided in the scope 1. Therefore, manufacturing costs of the scope 1 are reduced, and an operation of the scope 1 for a single use is facilitated.

### Internal Configuration of Scope 1 and Interface Adapter 4

FIG. 10 is a diagram showing an example of an internal configuration of the scope 1 and the interface adapter 4. As shown in FIG. 10, the distal end part 10C of the scope 1 is provided with an imaging optical system including an objective lens 21 and a lens group 22, an imaging sensor 23 that images a subject through the imaging optical system, a memory 25 such as a random-access memory (RAM), a communication interface (I/F) 26, an imaging drive unit 27, a light source device 5, and an illumination lens 50.

As the imaging sensor 23, a charge-coupled device (CCD) image sensor or a complementary metal-oxide-semiconductor (CMOS) image sensor is used. The imaging sensor 23 may perform imaging by a rolling shutter method or may perform imaging by a global shutter method.

The imaging sensor 23 has a light-receiving surface in which a plurality of pixels are two-dimensionally arranged, and converts an optical image formed on the light-receiving surface by the imaging optical system into an electrical signal (imaging signal) in each pixel. Then, the imaging sensor 23 converts the converted imaging signal from an analog signal into a digital signal having a predetermined number of bits, and outputs the imaging signal converted into the digital signal to the memory 25. For example, the imaging sensor 23 equipped with a color filter such as a primary color filter or a complementary color filter is used. A set of the imaging signals output from the respective pixels on the light-receiving surface of the imaging sensor 23 is referred to as a captured image signal.

The memory 25 temporarily records the digital imaging signal output from the imaging sensor 23. The communication interface (I/F) 26 is connected to a first communication interface (I/F) 41 of the interface adapter 4. The communication interface 26 transmits the imaging signal recorded in the memory 25 to the interface adapter 4 through a signal line in the communication cable 13.

The imaging drive unit 27 is connected to a system control unit 44 of the interface adapter 4 via the communication interface 26. The imaging drive unit 27 drives the imaging sensor 23 and the memory 25 based on a command from the system control unit 44 received by the communication interface 26.

The light source device 5 can emit, as illumination light, normal light having a light emission spectrum suitable for recognition by a human, such as a doctor, for example, white light. Furthermore, the light source device 5 may be able to emit, as the illumination light, special light having a light emission spectrum suitable for image analysis by a computer, such as image-enhanced endoscopy (IEE), which is different from the light emission spectrum of normal light. For example, a semiconductor light source is used as a light source of the light source device 5.

The light source device 5 is connected to the system control unit 44 of the interface adapter 4 via the communication interface 26. The light source device 5 emits the illumination light based on a command from the system control unit 44 received by the communication interface 26.

The illumination lens 50 irradiates an imaging target (for example, the inside of the subject) to be imaged by the imaging sensor 23 and the imaging optical system with the illumination light emitted from the light source device 5. An aperture may be included between the illumination lens 50 and the light source device 5 or in the imaging optical system, but from the viewpoint of reducing the manufacturing costs of the scope 1, it is preferable that no aperture is included. Since the scope 1 does not include an aperture, the exposure control of the scope I can be simplified, and thus manufacturing costs of the interface adapter 4 can be reduced.

The interface adapter 4 comprises the first communication interface 41 that is connected to the communication interface 26 of the scope 1 via the communication cable 13, the signal processing unit 42, a second communication interface (I/F) 43, and the system control unit 44.

The first communication interface 41 is for communicating with the scope 1, and has, for example, the video input terminal 4a shown in FIGS. 2 and 3 to receive the imaging signal transmitted from the communication interface 26 of the scope 1 via the communication cable 13 and transmit the imaging signal to the signal processing unit 42.

The signal processing unit 42 has a built-in memory such as a RAM that temporarily records the digital imaging signal received from the first communication interface 41, and performs processing (image processing such as amplification processing for amplifying each imaging signal, demosaicing processing, white balance processing, or gamma correction processing) on the captured image signal, which is a set of the imaging signals recorded in the memory, to generate image data in a format that can be displayed by the portable information terminal 7 which is a general-purpose terminal. In order to enable the white balance processing to be set for each imaging sensor, a white balance gain may be written into the memory mounted in the scope 1 at the time of manufacturing, and the white balance gain may be read out from the memory and used during the white balance processing. From the viewpoint of reducing the manufacturing costs, a fixed white balance gain may be used in the white balance processing.

The second communication interface 43 is for communicating with the portable information terminal 7, and has, for example, the video output terminal 4b shown in FIG. 3 and the like to transmit the image data generated by the signal processing unit 42 to the portable information terminal 7.

The system control unit 44 controls each unit of the interface adapter 4, sends a command to the scope 1, and performs overall control of the endoscope system 100. For example, the system control unit 44 is an example of a control unit of the imaging sensor 23 that controls the imaging by the imaging sensor 23 via the imaging drive unit 27. In addition, the system control unit 44 is an example of a control unit of the light source device 5 that controls the irradiation of the illumination light by the light source device 5. In the present embodiment, the system control unit 44 causes the imaging sensor 23 to perform imaging in a state in which a light amount of the illumination light to be emitted from the light source device 5 is controlled to be constant.

In addition, the system control unit 44 performs metering processing of deriving a metering value (hereinafter, referred to as brightness Y) indicating a brightness of image data based on the captured image signal obtained by the imaging sensor 23, and exposure control of controlling an imaging sensitivity of the imaging sensor 23 (specifically, an amplification factor of the imaging signal in the amplification processing) and an exposure time of the imaging sensor 23 based on the brightness Y obtained by the metering processing. For example, the system control unit 44 derives a brightness YA of the captured image signal based on the captured image signal before the amplification processing by the signal processing unit 42, amplifies the brightness YA by an amplification factor used in the amplification processing to derive a brightness Y, and controls the imaging sensitivity of the imaging sensor 23 and the exposure time of the imaging sensor 23 such that the brightness Y approaches a brightness (hereinafter, referred to as a target brightness Yt) suitable for recognition by a human, such as a doctor.

The signal processing unit 42 and the system control unit 44 each include various processors that execute programs to perform processing, a RAM, and a read-only memory (ROM). The various processors include a central processing unit (CPU) that is a general-purpose processor executing a program to perform various types of processing, a programmable logic device (PLD) that is a processor of which a circuit configuration can be changed after manufacture such as a field-programmable gate array (FPGA), or a dedicated electrical circuit that is a processor having a circuit configuration designed to be dedicated to executing specific processing such as an application-specific integrated circuit (ASIC). More specifically, a structure of these various processors is an electrical circuit in which circuit elements such as semiconductor elements are combined.

The signal processing unit 42 and the system control unit 44 may be configured with one of the various processors, or may be configured with a combination of two or more processors of the same type or different types (for example, a combination of a plurality of FPGAs or a combination of a CPU and an FPGA). It is preferable that the signal processing unit 42 and the system control unit 44 are configured with only the FPGA from the viewpoint of cost reduction and size reduction of the interface adapter 4.

### Hardware Configuration of Portable Information Terminal 7

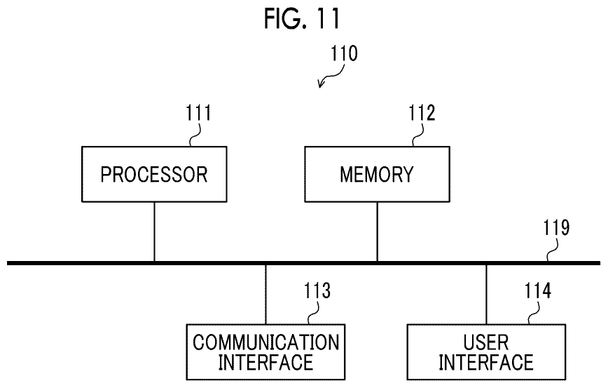

FIG. 11 is a diagram showing an example of a hardware configuration of the portable information terminal 7. The portable information terminal 7 can be implemented by, for example, a general-purpose information terminal 110 shown in FIG. 11. The information terminal 110 comprises a processor 111, a memory 112, a communication interface 113, and a user interface 114. The processor 111, the memory 112, the communication interface 113, and the user interface 114 are connected by, for example, a bus 119.

The processor 111 is a circuit that performs signal processing and is, for example, a central processing unit (CPU) that controls the entire information terminal 110. The processor 111 may be implemented by other digital circuits such as a field-programmable gate array (FPGA) and a digital signal processor (DSP). In addition, the processor 111 may be implemented by combining a plurality of digital circuits with each other.

The memory 112 includes, for example, a main memory and an auxiliary memory. The main memory is, for example, a RAM. The main memory is used as a work area of the processor 111.

The auxiliary memory is, for example, a non-volatile memory such as a magnetic disk, an optical disk, or a flash memory. The auxiliary memory stores various programs for operating the information terminal 110. The programs stored in the auxiliary memory are loaded into the main memory and executed by the processor 111.

In addition, the auxiliary memory may include a portable memory that can be detached from the information terminal 110. Examples of the portable memory include a USB flash drive and a memory card such as a secure digital (SD) memory card.

The communication interface 113 is a communication interface that performs communication with an outside of the information terminal 110 (for example, the interface adapter 4). For example, the communication interface 113 is a wired communication interface having a terminal that can be connected to the video output terminal 4b of the interface adapter 4 via a communication cable. Alternatively, the communication interface 113 may be a wireless communication interface that can perform wireless communication with the interface adapter 4. The communication interface 113 is controlled by the processor 111.

The user interface 114 includes, for example, an input device that receives an operation input from the user, and an output device that outputs information to the user. For example, the input device and the output device are implemented by the display unit 7a configured as a touch panel. In addition, the user interface 114 may include keys, a remote control, or the like as the input device. In addition, the user interface 114 may include a speaker, a vibrator, or the like as the output device. The user interface 114 is controlled by the processor 111.

For example, the touch panel included in the user interface 114 displays the image data received from the interface adapter 4 via the communication interface 113. Accordingly, an image based on the imaging signal obtained by the imaging sensor 23 of the scope 1 can be displayed to a user such as a doctor.

In addition, the touch panel included in the user interface 114 receives an instruction to perform imaging by the imaging sensor 23 of the scope 1 or an instruction to perform irradiation of illumination light by the light source device 5 of the scope 1 through a user operation. A control signal indicating the user operation received by the user interface 114 is transmitted to the system control unit 44 of the interface adapter 4 via the communication interface 113. The system control unit 44 performs control of imaging by the imaging sensor 23 of the scope 1 and control of irradiation of illumination light by the light source device 5 of the scope 1 based on the received control signal.

### Detailed Example of Metering Processing and Exposure Control

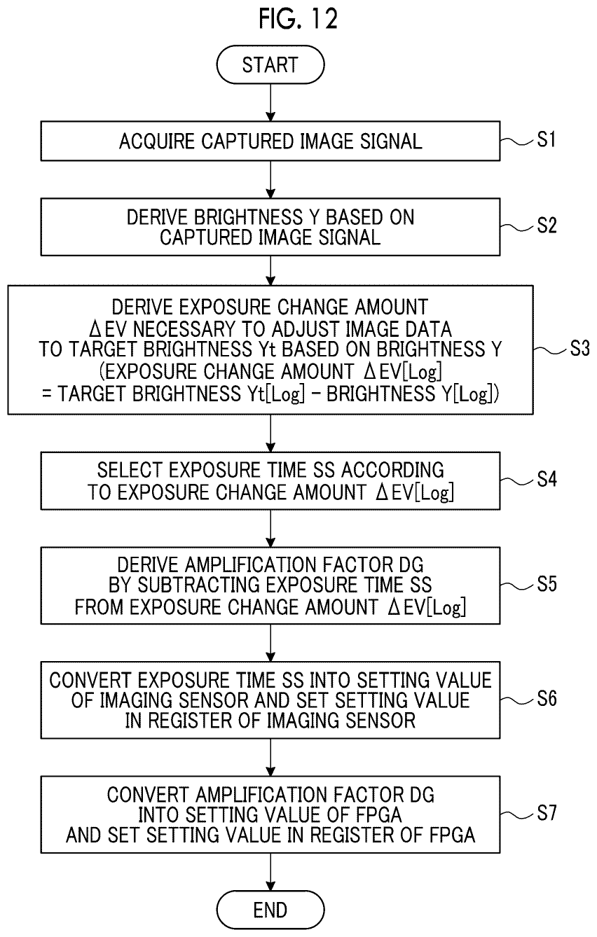

FIG. 12 is a flowchart for describing a detailed example of the metering processing and the exposure control performed by the system control unit 44. In the following, a detailed example of the metering processing and the exposure control will be described assuming a case where the system control unit 44 is configured with only an FPGA. In addition, in the following, a value obtained by converting each of a plurality of exposure times that can be set in the imaging sensor 23 into information in a logarithmic space by a logarithmic conversion lookup table (hereinafter, referred to as a first LUT) is referred to as an exposure time SS, and a value obtained by converting each of a plurality of amplification factors (digital gains) that can be set and used in the amplification processing performed by the signal processing unit 42 into information in a logarithmic space by the first LUT is referred to as an amplification factor DG. The first LUT is generated according to, for example, an expression y=Log(x)×a. In this expression, a is a predetermined coefficient, x is a numerical value before conversion, and y is a numerical value after conversion.

In a case where the system control unit 44 acquires the captured image signal before being subjected to the amplification processing by the signal processing unit 42 (step S1), the system control unit 44 performs a metering calculation on the captured image signal by a predetermined metering method to derive the brightness YA, and multiplies the brightness YA by the amplification factor during the amplification processing currently set to derive the brightness Y (step S2). The metering method is not particularly limited, but in the case of an endoscope, for example, a method of obtaining a metering value by changing weights for a central portion and a peripheral portion of the captured image signal can be preferably used. In this case, the calculation of Expression (1) is performed in order to derive an image feature amount necessary for deriving the metering value.

$$Image feature amount = \{ α \times central portion brightness value + ( 100 - α ) \times peripheral portion brightness value \} / 100 \qquad ( 1 )$$

In Expression (1), a is a coefficient for changing the weights for the central portion and the peripheral portion, and may be a fixed value determined on a system side or a variable value selected from a plurality of values depending on an imaging mode, preference of the user, and the like.

A division operation shown in Expression (1) can be replaced with an arithmetic operation that does not include division, as in Expression (2), by using a bit shift operation. Here, β is a coefficient corresponding to α.

$$\{ β \times central portion brightness value + ( 256 - β ) \times peripheral portion brightness value \} >> 8 \qquad ( 2 )$$

In a case where α of Formula (1) is, for example, 20, β of Formula (2) is 51.

In a case where α of Formula (1) is, for example, 40, β of Formula (2) is 102.

In a case where α of Formula (1) is, for example, 60, β of Formula (2) is 154.

In a case where α of Formula (1) is, for example, 80, β of Formula (2) is 205.

The system control unit 44 may derive the image feature amount by either Expression (1) or Expression (2). However, in a case where the system control unit 44 is configured only with the FPGA, it is preferable to derive the image feature amount by the arithmetic operation shown in Expression (2) in step S2 and to derive the brightness Y (metering value) by using the image feature amount. The arithmetic operation shown in Expression (2) does not include division, and is performed only by addition and multiplication. Therefore, it is possible to reduce a circuit scale of the FPGA or to improve a calculation speed of the FPGA.

After deriving the brightness Y in step S2, the system control unit 44 derives an exposure change amount ΔEV necessary to adjust the image data to the target brightness Yt based on the brightness Y (step S3). The exposure change amount ΔEV can be derived by, for example, an arithmetic operation of Expression (3).

$$Exposure change amount Δ EV = target brightness Yt \div brightness Y \qquad ( 3 )$$

Expression (3) can be replaced with an arithmetic expression that does not include division, as in Equation (4), by converting the target brightness Yt and the brightness Y into information in a logarithmic space. [Log] indicates information in a logarithmic space.

$$Exposure change amount Δ EV [ Log ] = target brightness Yt [ Log ] - brightness Y [ Log ] \qquad ( 4 )$$

The target brightness Yt[Log] is a value obtained by converting the target brightness Yt into information in a logarithmic space by the first LUT. The brightness Y[Log] is a value obtained by converting the brightness Y into information in a logarithmic space by the first LUT.

The arithmetic operation shown in Expression (4) does not include division and is performed only by subtraction. In other words, the arithmetic operation shown in Expression (4) is performed only by subtraction out of division and subtraction. Therefore, in a case where the system control unit 44 is configured only with the FPGA, it is possible to reduce the circuit scale of the FPGA or to improve the calculation speed of the FPGA.

Here, since it is assumed that the system control unit 44 is configured only with the FPGA, in step S3, the system control unit 44 converts the brightness Y into the brightness Y[Log] and converts the target brightness Yt into the target brightness Yt[Log] using the first LUT, and subtracts the brightness Y[Log] from the target brightness Yt[Log] to derive the exposure change amount ΔEV[Log]. The larger the value of the exposure change amount ΔEV[Log] is, the darker the image data is relative to the target brightness Yt.

Next, the system control unit 44 selects one of the plurality of exposure times SS according to a magnitude of the exposure change amount ΔEV[Log] (step S4). For example, the system control unit 44 divides the exposure change amounts ΔEV[Log], which are zero or greater, into a plurality of ranges according to the magnitudes thereof.

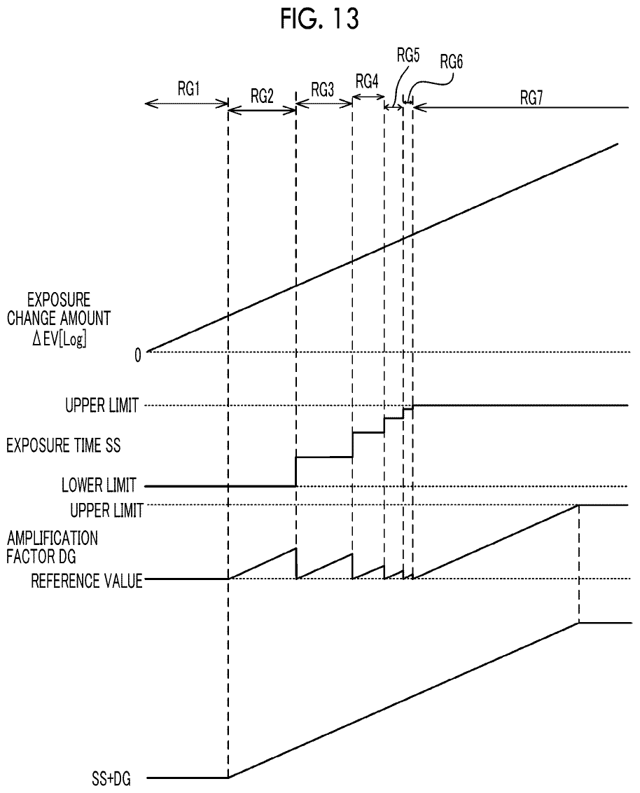

FIG. 13 is a schematic diagram for describing a relationship between the magnitude of the exposure change amount ΔEV[Log], the exposure time SS, and the amplification factor DG. In each graph of FIG. 13, numerical values increase in an upward direction along a vertical axis. FIG. 13 shows an example of a case where six exposure times SS can be set. In addition, in order to ensure that an observation image composed of continuously acquired image data appears natural, as an example, it is preferable that an upper limit value of the exposure time SS is set to ( 1/38) seconds and a lower limit value thereof is set to ( 1/800) seconds.

In the example of FIG. 13, a range RG1, a range RG2, a range RG3, a range RG4, a range RG5, a range RG6, and a range RG7 are set in order from the smallest exposure change amount ΔEV[Log]. In step S4, in a case where the exposure change amount ΔEV[Log] belongs to the range RG1 and the range RG2, the system control unit 44 selects the smallest value (lower limit value) among the six exposure times SS. In a case where the exposure change amount ΔEV[Log] belongs to the range RG3, the system control unit 44 selects the second smallest value among the six exposure times SS. In a case where the exposure change amount ΔEV[Log] belongs to the range RG4, the system control unit 44 selects the third smallest value among the six exposure times SS. In a case where the exposure change amount ΔEV[Log] belongs to the range RG5, the system control unit 44 selects the fourth smallest value among the six exposure times SS. In a case where the exposure change amount ΔEV[Log] belongs to the range RG6, the system control unit 44 selects the fifth smallest value among the six exposure times SS. In a case where the exposure change amount ΔEV[Log] belongs to the range RG7, the system control unit 44 selects the maximum value (upper limit value of the exposure time SS) among the six exposure times SS.

The range RG1 shown in FIG. 13 is a range in which the exposure cannot be further reduced, and is a range in which the brightness of the generated image data is equal to or greater than the target brightness Yt. In a case where the exposure change amount ΔEV[Log] belongs to the range RG1, the lower limit value is selected as the exposure time SS, and a reference value (for example, a value corresponding to 1×) is selected as the amplification factor DG.

After the exposure time SS is selected in step S4, the system control unit 44 derives the amplification factor DG by an arithmetic operation of Expression (5) (step S5).

$$Amplification factor DG = exposure change amount Δ EV [ Log ] - exposure time SS \qquad ( 5 )$$

In a case where each numerical value in Expression (5) is converted from information in a logarithmic space to information in a real space, Expression (6) is obtained.

$$Amplification factor DG [ real number ] = exposure change amount Δ EV [ real number ] - exposure time SS [ real number ] \qquad ( 6 )$$

In the present embodiment, since the light amount of the illumination light is constant and an exposure value is determined by multiplying the amplification factor by the exposure time, Expression (6) holds. Accordingly, in a case where each value in Expression (6) is converted into information in a logarithmic space, Expression (5) holds. Therefore, the amplification factor DG can be obtained by the arithmetic operation of Expression (5). As described above, in step S5, only subtraction out of division and subtraction is performed. Therefore, it is possible to reduce the circuit scale of the system control unit 44 and to improve a derivation speed of the amplification factor DG.

In the example of FIG. 13, in a case where the exposure change amount ΔEV[Log] is in any range from the range RG2 to the range RG7, the system control unit 44 determines the amplification factor DG to be a larger value as the exposure change amount ΔEV[Log] becomes larger. In addition, in the ranges RG2 to RG6, the amplification factor DG is changed within a range from the reference value to a predetermined value smaller than an upper limit value of the amplification factor DG. Furthermore, in the ranges RG2 to RG6, a variation range of the amplification factor DG (the difference between the reference value and the above-mentioned predetermined value) becomes larger as the range approaches the range RG1. On the other hand, in the range RG7, the amplification factor DG is changed within a range between the reference value and the upper limit value.

After deriving the amplification factor DG in step S5, the system control unit 44 converts the exposure time SS selected in step S4 into a setting value for the imaging sensor 23 by using the lookup table, and sets the setting value in a register of the imaging sensor 23 (step S6). Accordingly, in the next imaging frame, the exposure time of the imaging sensor 23 becomes a value corresponding to the exposure time SS selected in step S4.

In addition, the system control unit 44 converts the amplification factor DG derived in step S5 into a setting value for the signal processing unit 42 using the lookup table, and sets the setting value in a register of the signal processing unit 42 (step S7). Accordingly, in the next imaging frame, the amplification factor of the imaging signal during the amplification processing becomes a value corresponding to the amplification factor DG derived in step S5.

As shown in FIG. 13, the exposure time of the imaging sensor 23 is discretely changed based on the brightness Y of the image data, and the amplification factor during the amplification processing is continuously changed based on the brightness Y of the image data. By discretely changing the exposure time, the costs of the imaging sensor 23 and the costs for control thereof can be reduced. Meanwhile, since the amplification factor can be continuously changed, the brightness of the image data can be finely adjusted, and quality of the image displayed on the portable information terminal 7 can be improved.

In addition, in the example of FIG. 13, the maximum value of the amplification factor DG decreases as the exposure change amount ΔEV[Log] increases from the range RG2 to the range RG6. For example, in a case where the exposure change amount ΔEV[Log] is in the range RG6, the image data is dark, and increasing the amplification factor may result in increased visible noise. Therefore, in such a situation, a signal-to-noise ratio can be improved by reducing a change width of the amplification factor. In a case where the exposure change amount ΔEV[Log] is in the range RG7, the exposure time SS has reached the upper limit value, so that no further increase in the exposure can be achieved except by increasing the amplification factor DG. Therefore, by changing the amplification factor DG within a range between the reference value and the upper limit value, the image data can be brought closer to the target brightness even in a very dark imaging environment.

In the example of FIG. 13, it is assumed that the amplification factor DG changes in each of the ranges RG2 to RG6 according to the magnitude of the exposure change amount ΔEV[Log]. However, the present invention is not limited thereto. For example, in each of the ranges RG2 to RG6, the amplification factor DG may remain at the reference value (corresponding to 1×). In such a case, the signal-to-noise ratio can be improved over a wide range covering the ranges RG1 to RG6.

### Preferred Aspect of Image Processing

It is preferable that the signal processing unit 42 performs the above-described gamma correction processing to generate the image data such that an output value of the display unit 7a in a case where a pixel value equal to or less than a first threshold value TH1 is input to the display unit 7a is greater than the pixel value, and an output value of the display unit 7a in a case where a pixel value exceeding the first threshold value TH1 is input to the display unit 7a matches the pixel value.

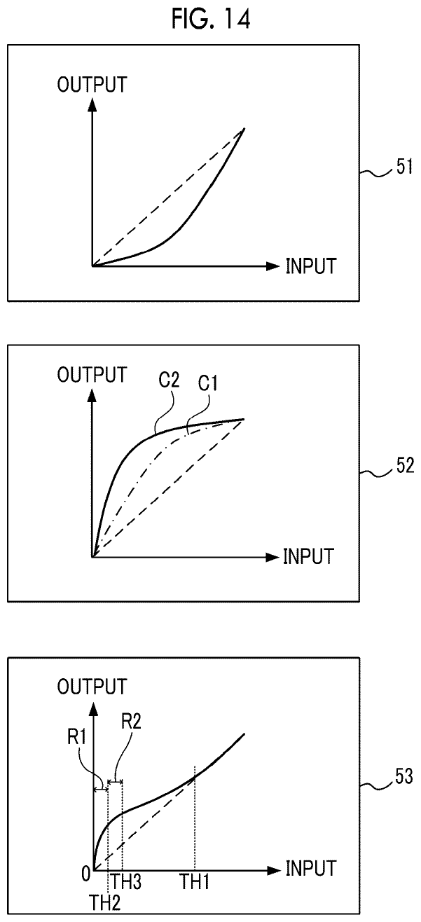

FIG. 14 is a schematic diagram for describing the gamma correction processing. A graph 51 shows gamma characteristics (for example, γ=2.2) of the display unit 7a. A straight line indicated by a broken line in the graph 51 indicates ideal characteristics in which an input and an output are in a direct proportion relationship. In general, in a case where the gamma correction is performed, the gamma characteristics of the graph 51 are taken into consideration, and the gamma correction is performed on image data before correction using correction data C1 shown in a graph 52 such that in a case where a pixel value of image data after correction is input to the display unit 7a, the pixel value and an output value (display brightness value) of the pixel value match each other.

In the present embodiment, for example, the gamma correction is performed on the image data before correction using correction data C2 shown in the graph 52. A graph 53 shows a relationship between a pixel value (input value) of image data obtained by performing the gamma correction according to the correction data C2 and an output value of the display unit 7a in a case where the pixel value is input to the display unit 7a.

As shown in the graph 53, output gradation characteristics of the image data after the gamma correction substantially match the straight line of the ideal characteristics in a range in which the pixel value exceeds the first threshold value TH1, and deviate upward from the straight line of the ideal characteristics in a range in which the pixel value is equal to or less than the first threshold value TH1. That is, in the image data after the gamma correction, the output value of the display unit 7a in a case where a pixel value equal to or less than the first threshold value TH1 is input to the display unit 7a is greater than the pixel value, and the output value of the display unit 7a in a case where a pixel value exceeding the first threshold value TH1 is input to the display unit 7a is in a state of matching the pixel value.

In the graph 53, a range from a minimum value (=0) to a second threshold value TH2 smaller than the first threshold value TH1 among the pixel values of the image data after the gamma correction is defined as a first range R1, and a range from a third threshold value TH3 that is between the first threshold value TH1 and the second threshold value TH2 to the second threshold value TH2 is defined as a second range R2, and these threshold values and ranges are shown.

In the image data after the gamma correction, a difference between an output value of the display unit 7a in a case where a pixel value in the second range R2 is input to the display unit 7a and the pixel value is greater than a difference between an output value of the display unit 7a in a case where a pixel value in the first range R1 is input to the display unit 7a and the pixel value. That is, the image data after the gamma correction has a very dark region (a region having a pixel value in the first range R1) with a very small pixel value and a slightly dark region (a region having a pixel value in the second range R2) with a slightly small pixel value, and displays the slightly dark region relatively brighter.

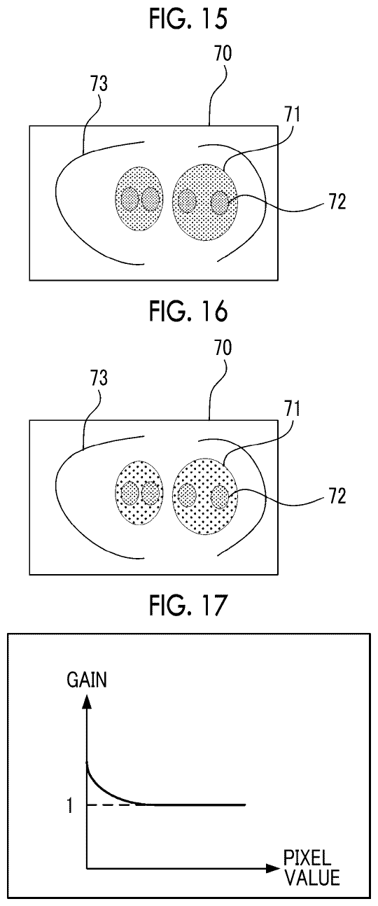

FIG. 15 is a schematic diagram showing an example of an image displayed on the display unit 7a in a case where the scope 1 is a bronchoscope. FIG. 15 shows an example in a case where the output gradation characteristics of the image data after the gamma correction are the ideal characteristics. A display image 70 shows a large tube 73, a small tube 71, and a branch tube 72 inside the small tube 71. A brightness of the small tube 71 is greater than that of the branch tube 72, but a difference between the two brightnesses is extremely small.

FIG. 16 is a diagram showing a display example of the image data subjected to the gamma correction to have the output gradation characteristics of the solid line shown in the graph 53. Between the small tube 71 and the branch tube 72, the small tube 71, which is relatively bright, is displayed brighter. As a result, a state of the branch tube 72 seen in the small tube 71 is easily visually recognized compared to FIG. 15.

The signal processing unit 42 does not need to use the correction data C2 of the graph 52 in order to generate the image data having the output gradation characteristics indicated by the solid line of the graph 53. For example, the gamma correction may be performed using the correction data C1, and then a gain greater than 1× may be applied to a small pixel value according to a gain correction table shown in FIG. 17, thereby obtaining the output gradation characteristics shown in the graph 53. More preferably, the gamma correction may be performed using the correction data C1, the pixel values may be converted into brightness values, and a gain greater than 1× may be applied to brightness values smaller than a predetermined value according to the gain correction table shown in FIG. 17, thereby obtaining the output gradation characteristics shown in the graph 53.

In the above description, the imaging sensitivity of the imaging sensor 23 is changed by changing the amplification factor used in the amplification processing performed by the signal processing unit 42. However, for example, a configuration may be adopted in which the imaging sensitivity is changed by changing the amplification factor of an amplifier that amplifies the analog signal included in the imaging sensor 23.

As described above, the following matters are disclosed in the present specification. Although corresponding constituents and the like in the embodiment described above are shown in parentheses, the present invention is not limited thereto.

(1)
An interface adapter comprising:
a first communication interface (first communication interface 41) for communicating with a scope (scope 1) including an imaging sensor (imaging sensor 23) and a light source device (light source device 5) that generates illumination light for imaging by the imaging sensor;
a second communication interface (second communication interface 43) for communicating with a portable information terminal; and
a processor (system control unit 44 and signal processing unit 42),
in which the processor
   causes the imaging sensor to perform imaging in a state in which a light amount of the illumination light is controlled to be constant,
   converts a captured image signal obtained by the imaging sensor into image data that is displayable by the portable information terminal, and transmits the image data to the portable information terminal,
   derives a brightness of the image data based on the captured image signal, and
   controls an imaging sensitivity of the imaging sensor and an exposure time of the imaging sensor based on the brightness.

According to (1), exposure control is performed by adjusting two parameters, the imaging sensitivity and the exposure time, in a state in which the amount of illumination light is constant. Since the number of adjustment targets for the exposure control is narrowed down to two, the processor does not need to have advanced capabilities. As a result, a system in which an image obtained by imaging with a scope can be displayed and checked by a general-purpose portable information terminal connected by the second communication interface can be implemented at a low cost. In addition, it is possible to reduce a size and a weight of the interface adapter.

(2)
The interface adapter according to (1),
in which the processor increases, in a case where the exposure time is controlled to an upper limit value (a case where the exposure change amount ΔEV[Log] is in the range RG7), a variation range of the imaging sensitivity compared to a case where the exposure time is controlled to be less than the upper limit value (a case where the exposure change amount ΔEV[Log] is in the ranges RG2 to RG6).

According to (2), the variation range of the imaging sensitivity is small until the exposure time reaches the upper limit value, so that a signal-to-noise ratio of the imaging signal can be improved. Meanwhile, after the exposure time reaches the upper limit value, the variation range of the imaging sensitivity can be increased, and thus a dark subject can be imaged brightly.

(3)
The interface adapter according to (2),
in which the processor discretely changes the exposure time and continuously changes the imaging sensitivity.

According to (3), since an imaging sensor with advanced capabilities (an imaging sensor capable of finely controlling an exposure time) is not required, it is possible to reduce the manufacturing costs of the scope and to reduce the manufacturing costs of the interface adapter by simplifying control contents of the scope, and it is possible to reduce construction costs of the system.

(4)
The interface adapter according to (2),
in which the processor sets the exposure time to a different value for each of a plurality of ranges of the brightness (each range in which the exposure change amount ΔEV[Log] is in the ranges RG2 to RG7), and changes the imaging sensitivity within a range from a reference value (amplification factor DG=1×) to a value lower than an upper limit value of the imaging sensitivity in accordance with the brightness in a state in which the exposure time is controlled to be less than the upper limit value.

According to (4), since an imaging sensor with advanced capabilities (an imaging sensor capable of finely controlling an exposure time) is not required, it is possible to reduce the manufacturing costs of the scope and to reduce the manufacturing costs of the interface adapter by simplifying control contents of the scope, and it is possible to reduce the construction costs of the system. In addition, even in a state in which any exposure time is set, the imaging sensitivity can be changed to finely adjust the exposure according to the subject, thereby improving the quality of the image data.

(5)
The interface adapter according to any one of (1) to (4),
in which the processor performs amplification processing of amplifying the captured image signal, which is digital, output from the imaging sensor, and
the imaging sensitivity is an amplification factor set by the amplification processing.

According to (5), since the brightness of the image data can be controlled by amplifying the digital signal, the exposure control can be performed with high accuracy compared to a case where the amplification factor in a case of amplifying an analog signal inside the imaging sensor is controlled. In addition, since an imaging sensor having high analog signal amplification performance is not required, it is possible to reduce the manufacturing costs of the scope.

(6)
The interface adapter according to any one of (1) to (5),
in which the processor is a programmable logic device.

According to (6), it is possible to reduce the costs of the interface adapter and to reduce the construction costs of the system.

(7)
The interface adapter according to (6),
in which the processor performs, among division and subtraction, only the subtraction as arithmetic processing required to determine a combination of the imaging sensitivity and the exposure time for bringing the brightness (brightness Y) close to a target brightness (target brightness Yt).

According to (7), since the division is not performed to determine the imaging sensitivity and the exposure time, it is possible to reduce a circuit scale of the processor and to reduce a calculation time. As a result, it is possible to reduce the manufacturing costs of the interface adapter.

(8)
The interface adapter according to (7),
in which a value obtained by logarithmically converting each of a plurality of the exposure times settable in the imaging sensor is set as an exposure time logarithmic value (exposure time SS), and
the processor
   derives a first subtraction value (exposure change amount ΔEV[Log]) by subtracting a logarithmic conversion value (brightness Y[Log]) of the brightness from a logarithmic conversion value (target brightness Yt[Log]) of the target brightness,
   selects one from among a plurality of the exposure time logarithmic values according to a magnitude of the first subtraction value,
   derives a second subtraction value (amplification factor DG[Log]) by subtracting the selected exposure time logarithmic value from the first subtraction value, and
   converts the selected exposure time logarithmic value into a setting value of the exposure time using a conversion table (lookup table), and converts the second subtraction value into a setting value of the imaging sensitivity using a conversion table (lookup table).

According to (8), since division processing is not performed to determine the imaging sensitivity and the exposure time, it is possible to reduce the circuit scale of the processor and to reduce the calculation time.

(9)
The interface adapter according to any one of (6) to (8),
in which the processor performs an arithmetic operation required to determine the brightness by a bit shift operation.

According to (9), since division processing is not required to determine the brightness, it is possible to reduce the circuit scale of the processor and to reduce the calculation time.

(10)
The interface adapter according to any one of (1) to (9),
in which the processor
   performs, as processing of converting the captured image signal into the image data, gamma correction processing based on gamma characteristics of a display unit (display unit 7a) of the portable information terminal, and
   generates, in the gamma correction processing, the image data such that an output value of the display unit in a case where a pixel value equal to or less than a first threshold value (first threshold value TH1) is input to the display unit is greater than the pixel value, and an output value of the display unit in a case where a pixel value exceeding the first threshold value is input to the display unit matches the pixel value.

According to (10), in a case where a specific portion of the image captured by the imaging sensor is dark, for example, it is possible to brighten the dark portion regardless of gradation characteristics of a display device. Therefore, examination using the scope can be efficiently and accurately performed.

(11)
The interface adapter according to (10),
in which, in a case where, among pixel values of the image data, a range from a minimum value to a second threshold value (second threshold value TH2) smaller than the first threshold value is defined as a first range (first range R1) and a range from a third threshold value (third threshold value TH3) that is between the first threshold value and the second threshold value to the second threshold value is defined as a second range (second range R2),
   the processor generates, in the gamma correction processing, the image data such that a difference between an output value of the display unit in a case where a pixel value in the second range is input to the display unit and the pixel value is greater than a difference between an output value of the display unit in a case where a pixel value in the first range is input to the display unit and the pixel value.

According to (11), in a case where a plurality of regions that are dark regions in the image data and that have a slight difference in brightness are present, a brighter region among the plurality of regions is displayed brighter. Therefore, boundaries between the plurality of regions are easily visually recognized. For example, in a case where a bronchoscope is used and a trachea branches into two at the front, two branching points can be clearly visually recognized.

(12)
The interface adapter according to any one of (1) to (11),
in which the interface adapter is fixed to a frame (frame 8) capable of accommodating the portable information terminal.
   (13)
An endoscope system (endoscope system 100) comprising:
the interface adapter according to any one of (1) to (11);
a frame (frame 8) capable of accommodating the portable information terminal; and
the scope,
in which the interface adapter is fixed to the frame.

Although various embodiments have been described above, it goes without saying that the present invention is not limited to these examples. It is apparent that those skilled in the art may perceive various modification examples or correction examples within the scope disclosed in the claims, and those examples are also understood as falling within the technical scope of the present invention. In addition, each constituent in the embodiment may be used in any combination without departing from the gist of the invention.

The present application is based on Japanese Patent Application (JP2023-025924) filed on Feb. 22, 2023, the content of which is incorporated in the present application by reference.

### EXPLANATION OF REFERENCES

1: scope
4: interface adapter
4a: video input terminal
4b: video output terminal
5: light source device
7: portable information terminal
7a: display unit
8: frame
10: insertion part
10A: flexible part
10B: bending part
10C: distal end part
11: operation part
13: communication cable
22: lens group
23: imaging sensor
25, 112: memory
26, 113: communication interface
41: first communication interface
27: imaging drive unit
42: signal processing unit
43: second communication interface
44: system control unit
50: illumination lens
81: front cover
81a: opening portion
82: rear case
82a: cable insertion hole cover
82b: notation viewing hole
82c: stand
100: endoscope system
110: information terminal
111: processor
114: user interface
119: bus
RG1 to RG7: range
51, 52, 53: graph
C1, C2: correction data
R1: first range
R2: second range
70: display image
71: small tube
72: branch tube
73: large tube

What is claimed is:

1. An interface adapter comprising:
a first communication interface for communicating with a scope including an imaging sensor and a light source device that generates illumination light for imaging by the imaging sensor;
a second communication interface for communicating with a portable information terminal having a user interface; and
a processor,
wherein the processor
causes the imaging sensor to perform imaging in a state in which a light amount of the illumination light is controlled to be constant,
converts a captured image signal obtained by the imaging sensor into image data that is displayable by the portable information terminal, and transmits the image data to the portable information terminal,
derives a brightness of the image data based on the captured image signal, and
controls an imaging sensitivity of the imaging sensor and an exposure time of the imaging sensor based on the brightness, and
the interface adapter is controlled by the user interface of the portable information terminal.

2. The interface adapter according to claim 1,
wherein the processor increases, in a case where the exposure time is controlled to an upper limit value, a variation range of the imaging sensitivity compared to a case where the exposure time is controlled to be less than the upper limit value.

3. The interface adapter according to claim 2,
wherein the processor discretely changes the exposure time and continuously changes the imaging sensitivity.

4. The interface adapter according to claim 2,
wherein the processor sets the exposure time to a different value for each of a plurality of ranges of the brightness, and changes the imaging sensitivity within a range from a reference value to a value lower than an upper limit value of the imaging sensitivity in accordance with the brightness in a state in which the exposure time is set to be less than the upper limit value.

5. The interface adapter according to claim 1,
wherein the processor performs amplification processing of amplifying the captured image signal, which is digital, output from the imaging sensor, and
the imaging sensitivity is an amplification factor set by the amplification processing.

6. The interface adapter according to claim 1,
wherein the processor is a programmable logic device.

7. The interface adapter according to claim 6,
wherein the processor performs, among division and subtraction, only the subtraction as arithmetic processing required to determine a combination of the imaging sensitivity and the exposure time for bringing the brightness close to a target brightness.

8. The interface adapter according to claim 7,
wherein a value obtained by logarithmically converting each of a plurality of the exposure times settable in the imaging sensor is set as an exposure time logarithmic value, and
the processor
derives a first subtraction value by subtracting a logarithmic conversion value of the brightness from a logarithmic conversion value of the target brightness,
selects one from among a plurality of the exposure time logarithmic values according to a magnitude of the first subtraction value,
derives a second subtraction value by subtracting the selected exposure time logarithmic value from the first subtraction value, and
converts the selected exposure time logarithmic value into a setting value of the exposure time using a conversion table, and converts the second subtraction value into a setting value of the imaging sensitivity using a conversion table.

9. The interface adapter according to claim 6,
wherein the processor performs an arithmetic operation required to determine the brightness by a bit shift operation.

10. The interface adapter according to claim 1,
wherein the processor
performs, as processing of converting the captured image signal into the image data, gamma correction processing based on gamma characteristics of a display unit of the portable information terminal, and
generates, in the gamma correction processing, the image data such that an output value of the display unit in a case where a pixel value equal to or less than a first threshold value is input to the display unit is greater than the pixel value, and an output value of the display unit in a case where a pixel value exceeding the first threshold value is input to the display unit matches the pixel value.

11. The interface adapter according to claim 10,
wherein, in a case where, among pixel values of the image data, a range from a minimum value to a second threshold value smaller than the first threshold value is defined as a first range and a range from a third threshold value that is between the first threshold value and the second threshold value to the second threshold value is defined as a second range,
the processor generates, in the gamma correction processing, the image data such that a difference between an output value of the display unit in a case where a pixel value in the second range is input to the display unit and the pixel value is greater than a difference between an output value of the display unit in a case where a pixel value in the first range is input to the display unit and the pixel value.

12. The interface adapter according to claim 1,
wherein the interface adapter is fixed to a frame capable of accommodating the portable information terminal.

13. An endoscope system comprising:
the interface adapter according to claim 1;
a frame capable of accommodating the portable information terminal; and
the scope,
wherein the interface adapter is fixed to the frame.
